(19) United States
(12) Patent Application Publication
(10) Pub. No.: US 2022/0189626 A1
(43) Pub. Date: Jun. 16, 2022

(54) SYSTEMS AND METHODS FOR DETECTING AND ADDRESSING QUALITY ISSUES IN REMOTE THERAPY SESSIONS

(71) Applicant: Advanced Neuromodulation Systems, Inc., Plano, TX (US)

(72) Inventors: Scott DeBates, Frisco, TX (US); Tucker Tomlinson, Sachse, TX (US); Andrew Csavoy, Dallas, TX (US); Yagna Pathak, Skokie, IL (US); Lonnie Smith, Aubrey, TX (US); James Nagle, Celina, TX (US)

(21) Appl. No.: 17/370,247

(22) Filed: Jul. 8, 2021

Related U.S. Application Data
(60) Provisional application No. 63/124,404, filed on Dec. 11, 2020.

(51) Int. Cl. G16H 40/67; G16H 80/00; G06N 20/00; H04N 7/15

(52) U.S. Cl. CPC G16H 40/67; H04N 7/152; G06N 20/00; G16H 80/00

(57) ABSTRACT

The present disclosure provides systems and methods for improving quality of a remote therapy session. A method includes capturing, using a computing device, video data associated with a remote therapy session between a patient device and a clinician device, applying, using the computing device, one or more machine learning algorithms to the captured video data to detect a quality issue associated with the remote therapy session, the quality issue related to one of i) a field of view associated with the video data, ii) a luminance associated with the video data, and iii) a contrast associated with the video data, and performing, using the computing device, a remedial action to address the detected quality issue in the video data.

### CROSS-REFERENCE TO RELATED APPLICATIONS

This application claims priority to provisional application Ser. No. 63/124,404, filed Dec. 11, 2020, which is incorporated herein by reference in its entirety.

### A. FIELD OF THE DISCLOSURE

The present disclosure relates generally to remote therapy, and more particularly to detecting and addressing quality issues during a remote therapy session.

### B. BACKGROUND ART

Implantable medical devices have changed how medical care is provided to patients having a variety of chronic illnesses and disorders. For example, implantable cardiac devices improve cardiac function in patients with heart disease by improving quality of life and reducing mortality rates. Further, types of implantable neurostimulators provide a reduction in pain for chronic pain patients and reduce motor difficulties in patients with Parkinson's disease and other movement disorders. In addition, a variety of other medical devices currently exist or are in development to treat other disorders in a wide range of patients.

Many implantable medical devices and other personal medical devices are programmed by a physician or other clinician to optimize the therapy provided by a respective device to an individual patient. The programming may occur using short-range communication links (e.g., inductive wireless telemetry) in an in-person or in-clinic setting.

However, remote patient therapy is a healthcare delivery method that aims to use technology to manage patient health outside of a traditional clinical setting. It is widely expected that remote patient care may increase access to care and decrease healthcare delivery costs.

Notably, telehealth technology, such as remote patient therapy technology, is showing increasing adoption given its ability to address multiple challenges for patients and clinicians. For example, telehealth technology reduces travel burden and potential exposure to infections agents or hazards that may be present in an in-person clinical setting.

### BRIEF SUMMARY OF THE DISCLOSURE

In one embodiment, the present disclosure is directed to a method for improving quality of a remote therapy session. The method includes capturing, using a computing device, video data associated with a remote therapy session between a patient device and a clinician device, applying, using the computing device, one or more machine learning algorithms to the captured video data to detect a quality issue associated with the remote therapy session, the quality issue related to one of i) a field of view associated with the video data, ii) a luminance associated with the video data, and iii) a contrast associated with the video data, and performing, using the computing device, a remedial action to address the detected quality issue in the video data.

In another embodiment, the present disclosure is directed to a computing device for improving quality of a remote therapy session. The computing device includes a memory device, and a processor communicatively coupled to the memory device. The processor is configured to capture data associated with a remote therapy session between a patient device and a clinician device, apply one or more machine learning algorithms to the captured data to detect a quality issue associated with the remote therapy session, and perform a remedial action to address the detected quality issue.

In yet another embodiment, the present disclosure is directed to non-transitory computer-readable media having computer-executable instructions thereon. When executed by a processor of a computing device, the instructions cause the processor of the computing device to capture data associated with a remote therapy session between a patient device and a clinician device, apply one or more machine learning algorithms to the captured data to detect a quality issue associated with the remote therapy session, and perform a remedial action to address the detected quality issue.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a diagram of one embodiment of a network environment for implementing remote therapy sessions.

FIG. 2 shows one embodiment of a user interface that may be used within the network environment shown in FIG. 1.

FIG. 3 shows one embodiment of a user interface that may be used within the network environment shown in FIG. 1.

FIG. 4 shows one embodiment of a user interface that may be used within the network environment shown in FIG. 1.

FIG. 5 shows one embodiment of a user interface that may be used within the network environment shown in FIG. 1.

FIG. 6 is a block diagram of one embodiment of a computing device.

Corresponding reference characters indicate corresponding parts throughout the several views of the drawings.

### DETAILED DESCRIPTION OF THE DISCLOSURE

The present disclosure provides systems and methods for improving quality of a remote therapy session. A method includes capturing data associated with a remote therapy session between a patient device and a clinician device, applying one or more machine learning algorithms to the captured data to detect a quality issue associated with the remote therapy session, and performing a remedial action to address the detected quality issue.

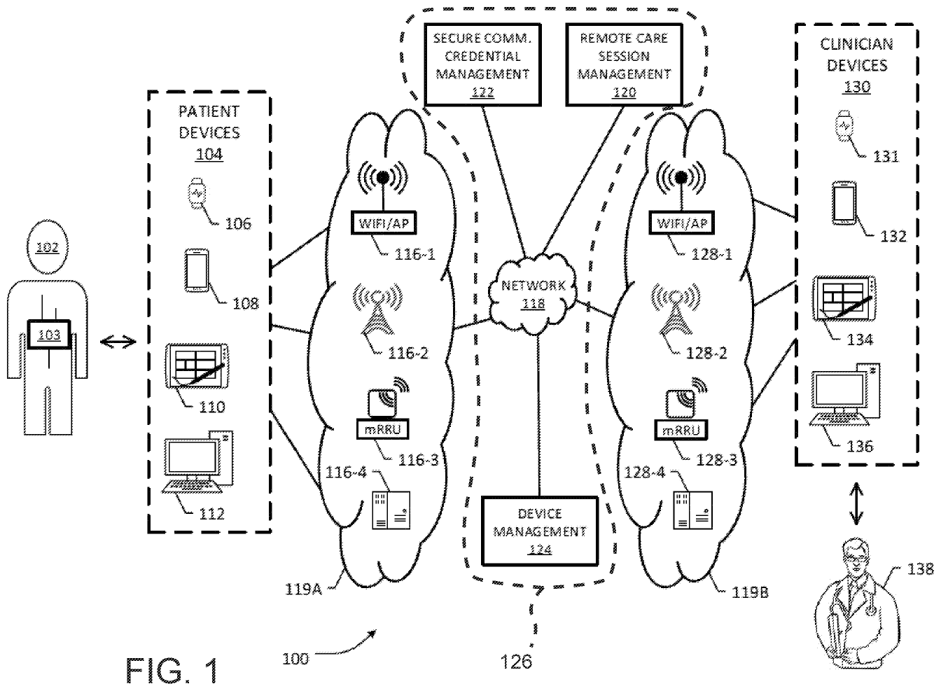

Referring now to the drawings, and in particular to FIG. 1, a network environment is indicated generally at 100. One or more embodiments of a remote care therapy application or service may be implemented in network environment 100, as described herein. In general, “remote care therapy” may involve any care, biomedical monitoring, or therapy that may be provided by a clinician, a medical professional or a healthcare provider, and/or their respective authorized agents (including digital/virtual assistants), with respect to a patient over a communications network while the patient and the clinician/provider are not in close proximity to each other (e.g., not engaged in an in-person office visit or consultation). Accordingly, in some embodiments, a remote care therapy application may form a telemedicine or a telehealth application or service that not only allows healthcare professionals to use electronic communications to evaluate, diagnose and treat patients remotely, thereby facilitating efficiency as well as scalability, but also provides patients with relatively quick and convenient access to diversified medical expertise that may be geographically distributed over large areas or regions, via secure communications channels as described herein.

Network environment 100 may include any combination or sub-combination of a public packet-switched network infrastructure (e.g., the Internet or worldwide web, also sometimes referred to as the “cloud”), private packet-switched network infrastructures such as Intranets and enterprise networks, health service provider network infrastructures, and the like, any of which may span or involve a variety of access networks, backhaul and core networks in an end-to-end network architecture arrangement between one or more patients, e.g., patient(s) 102, and one or more authorized clinicians, healthcare professionals, or agents thereof, e.g., generally represented as caregiver(s) or clinician(s) 138.

Example patient(s) 102, each having a suitable implantable device 103, may be provided with a variety of corresponding external devices for controlling, programming, otherwise (re)configuring the functionality of respective implantable medical device(s) 103, as is known in the art. Such external devices associated with patient(s) 102 are referred to herein as patient devices 104, and may include a variety of user equipment (UE) devices, tethered or untethered, that may be configured to engage in remote care therapy sessions. By way of example, patient devices 104 may include smartphones, tablets or phablets, laptops/desktops, handheld/palmtop computers, wearable devices such as smart glasses and smart watches, personal digital assistant (PDA) devices, smart digital assistant devices, etc., any of which may operate in association with one or more virtual assistants, smart home/office appliances, smart TVs, virtual reality (VR), mixed reality (MR) or augmented reality (AR) devices, and the like, which are generally exemplified by wearable device(s) 106, smartphone(s) 108, tablet(s)/phablet(s) 110 and computer(s) 112. As such, patient devices 104 may include various types of communications circuitry or interfaces to effectuate wired or wireless communications, short-range and long-range radio frequency (RF) communications, magnetic field communications, Bluetooth communications, etc., using any combination of technologies, protocols, and the like, with external networked elements and/or respective implantable medical devices 103 corresponding to patient(s) 102.

With respect to networked communications, patient devices 104 may be configured, independently or in association with one or more digital/virtual assistants, smart home/premises appliances and/or home networks, to effectuate mobile communications using technologies such as Global System for Mobile Communications (GSM) radio access network (GRAN) technology, Enhanced Data Rates for Global System for Mobile Communications (GSM) Evolution (EDGE) network (GERAN) technology, 4G Long Term Evolution (LTE) technology, Fixed Wireless technology, 5th Generation Partnership Project (5GPP or 5G) technology, Integrated Digital Enhanced Network (IDEN) technology, WiMAX technology, various flavors of Code Division Multiple Access (CDMA) technology, heterogeneous access network technology, Universal Mobile Telecommunications System (UMTS) technology, Universal Terrestrial Radio Access Network (UTRAN) technology, All-IP Next Generation Network (NGN) technology, as well as technologies based on various flavors of IEEE 802.11 protocols (e.g., WiFi), and other access point (AP)-based technologies and microcell-based technologies such as femtocells, picocells, etc. Further, some embodiments of patient devices 104 may also include interface circuitry for effectuating network connectivity via satellite communications. Where tethered UE devices are provided as patient devices 104, networked communications may also involve broadband edge network infrastructures based on various flavors of Digital Subscriber Line (DSL) architectures and/or Data Over Cable Service Interface Specification (DOCSIS)-compliant Cable Modem Termination System (CMTS) network architectures (e.g., involving hybrid fiber-coaxial (HFC) physical connectivity). Accordingly, by way of illustration, an edge/access network portion 119A is exemplified with elements such as WiFi/AP node(s) 116-1, macro/microcell node(s) 116-2 and 116-3 (e.g., including micro remote radio units or RRUs, base stations, eNB nodes, etc.) and DSL/CMTS node(s) 116-4.

Similarly, clinicians 138 may be provided with a variety of external devices for controlling, programming, otherwise (re)configuring or providing therapy operations with respect to one or more patients 102 mediated via respective implantable medical device(s) 103, in a local therapy session and/or remote therapy session, depending on implementation and use case scenarios. External devices associated with clinicians 138, referred to herein as clinician devices 130, may include a variety of UE devices, tethered or untethered, similar to patient devices 104, which may be configured to engage in remote care therapy sessions as will be set forth in detail further below. Clinician devices 130 may therefore also include devices (which may operate in association with one or more virtual assistants, smart home/office appliances, VRAR virtual reality (VR) or augmented reality (AR) devices, and the like), generally exemplified by wearable device(s) 131, smartphone(s) 132, tablet(s)/phablet(s) 134 and computer(s) 136. Further, example clinician devices 130 may also include various types of network communications circuitry or interfaces similar to that of patient device 104, which may be configured to operate with a broad range of technologies as set forth above. Accordingly, an edge/access network portion 119B is exemplified as having elements such as WiFi/AP node(s) 128-1, macro/microcell node(s) 128-2 and 128-3 (e.g., including micro remote radio units or RRUs, base stations, eNB nodes, etc.) and DSL/CMTS node(s) 128-4. It should therefore be appreciated that edge/access network portions 119A, 119B may include all or any subset of wireless communication means, technologies and protocols for effectuating data communications with respect to an example embodiment of the systems and methods described herein.

In one arrangement, a plurality of network elements or nodes may be provided for facilitating a remote care therapy service involving one or more clinicians 138 and one or more patients 102, wherein such elements are hosted or otherwise operated by various stakeholders in a service deployment scenario depending on implementation (e.g., including one or more public clouds, private clouds, or any combination thereof). In one embodiment, a remote care session management node 120 is provided, and may be disposed as a cloud-based element coupled to network 118, that is operative in association with a secure communications credentials management node 122 and a device management node 124, to effectuate a trust-based communications overlay/tunneled infrastructure in network environment 100 whereby a clinician may advantageously engage in a remote care therapy session with a patient.

In the embodiments described herein, implantable medical device 103 may be any suitable medical device. For example, implantable medical device may be a neurostimulation device that generates electrical pulses and delivers the pulses to nervous tissue of a patient to treat a variety of disorders.

One category of neurostimulation systems is deep brain stimulation (DBS). In DBS, pulses of electrical current are delivered to target regions of a subject's brain, for example, for the treatment of movement and effective disorders such as PD and essential tremor. Another category of neurostimulation systems is spinal cord stimulation (SCS) for the treatment of chronic pain and similar disorders.

Neurostimulation systems generally include a pulse generator and one or more leads. A stimulation lead includes a lead body of insulative material that encloses wire conductors. The distal end of the stimulation lead includes multiple electrodes, or contacts, that intimately impinge upon patient tissue and are electrically coupled to the wire conductors. The proximal end of the lead body includes multiple terminals (also electrically coupled to the wire conductors) that are adapted to receive electrical pulses. In DBS systems, the distal end of the stimulation lead is implanted within the brain tissue to deliver the electrical pulses. The stimulation leads are then tunneled to another location within the patient's body to be electrically connected with a pulse generator or, alternatively, to an “extension.” The pulse generator is typically implanted in the patient within a subcutaneous pocket created during the implantation procedure.

The pulse generator is typically implemented using a metallic housing (or can) that encloses circuitry for generating the electrical stimulation pulses, control circuitry, communication circuitry, a rechargeable battery, etc. The pulse generating circuitry is coupled to one or more stimulation leads through electrical connections provided in a “header” of the pulse generator. Specifically, feedthrough wires typically exit the metallic housing and enter into a header structure of a moldable material. Within the header structure, the feedthrough wires are electrically coupled to annular electrical connectors. The header structure holds the annular connectors in a fixed arrangement that corresponds to the arrangement of terminals on the proximal end of a stimulation lead.

Although implantable medical device 103 is described in the context of a neurostimulation device herein, those of skill in the art will appreciate that implantable medical device 103 may be any type of implantable medical device. Further, those of skill in the art will appreciate that the systems and methods described herein may be implemented in remote therapy sessions (e.g., telehealth sessions) that involve remotely programming an implantable medical device, as well as remote therapy sessions that do not involve remotely programming an implantable medical device.

As explained in more detail below, the systems and methods described herein assist users in improving the quality of remote therapy sessions by i) detecting potential quality issues with a remote therapy session (either in real-time during the session, or prior to the session), and ii) facilitating addressing any potential quality issues by, for example, notifying one or more users, providing feedback on the severity of the issues, and/or providing guidance on potential solutions. Further, in the embodiments described herein, machine learning and computational algorithms may be leveraged to reproduce clinically relevant metrics, and apply those clinically relevant metrics to improve quality of remote therapy sessions.

Although at least some of the example provided herein relate to remote therapy sessions involving deep brain stimulation, those of skill in the art will appreciate that the embodiments described herein are applicable to remote therapy sessions for patient with other implantable devices (e.g., neurostimulators for chronic pain, or drug delivery pumps), as well as remote therapy sessions that are purely evaluative (e.g., for patients without active implants, or patients with implanted devices that are not remotely controllable).

One aspect of remote therapy, or telehealth sessions distinct from standard teleconferencing calls is the need for a clinician (e.g., clinician 138) to evaluate the status of a patient (e.g., patient 102). This need is important to the utility of telehealth technologies as a component of modern healthcare. This leads to a number of potential requirements that are not present in a standard teleconference session. For example, the clinician generally needs to be able to clearly see the patient. This ability may turn on both the quality of the video feed (which must be of sufficient quality for the clinician to accurately observe the patient), as well as the field of view acquired by the patient device (e.g., patient device 104) (which should must include the affected area of the body being assessed by the clinician).

As described herein, in addition to threshold detection methods (e.g., based on ambience luminance or background noise detection), machine learning systems may be trained to detect specific features of interest on the patient and determine when they are not clearly depicted. Notably, machine learning systems provide not only detection, but also measures of certainty in detection classification. These measures of certainty may be used as proxies for clarity of the features of interest. Further, machine learning systems that detect features of interest may allow for more targeted applications of threshold based problem detection. Additionally, machine learning systems may be employed to directly detect specific scenarios that impair remote therapy session quality (e.g., backlighting and/or dim lighting impacting video quality, and background noise impacting audio quality).

Using the embodiments described herein, quality assessments and resulting system behaviors may be conducted prior to initiation of a remote therapy session, providing users with an opportunity to address issues prior to the session. Additionally or alternatively, quality assessments may be made periodically or continuously during the remote therapy session, alerting one or more users if issues arise, and/or triggering one or more devices to directly address detected issues.

The following provides several examples of improving quality of remote therapy sessions. In the examples described herein, a device (e.g., patient device 104 and/or clinician device 130) generally acquires data during a remote therapy sessions, assesses that data to detect at least one quality issue, and takes a remedial action to attempt to address the at least one quality issue.

Field of View

A telehealth system (e.g., implemented in network environment 100) typically includes a video interface that allows participants (e.g., a clinician and a patient) to see one another. One limitation of using such a video interface for the assessment of symptoms is that the field of view (e.g., acquired by patient device 104) may be inadequate to observe or focus on specific patient features that are of interest.

Accordingly, in one embodiment, a machine learning algorithm is trained to identify a feature of interest, and determines when acquired video data shows the feature of interest. For example, a remote therapy session software application operation on a patient device may analyze acquired video data to determine whether the feature of interest is shown.

If the feature of interest is not shown, the system generates a notification (e.g., on a user interface of the patient device) to alert the patient that the field of view should be adjusted (e.g., by adjusting a zoom setting or orientation of the field of view). Further, the user interface may highlight a portion of a displayed video to indicate where the obscured feature of interest is located, and/or may provide other cues (e.g., color-based cues) to assist the patient in adjusting the field of view.

In one example, detection of features such as faces or hands enables identifying the portion of the displayed video that these features constitute, allowing for both direct feedback and indirect assistive aid. Direct feedback may include notifying the patient that they are too far away from the patient device, and identifying which body part is not visible (e.g., displaying the following message to the patient “Your hands are difficult to see, please move closer to the camera”).

If the system has control over the camera, in some embodiments, the camera may be controlled (e.g., by the remote therapy application) to automatically zoom and crop the displayed video to provide improved visibility of the feature of interest. Alternatively, or additionally, a user interface may be provided on a remote computing device (e.g., a clinician device communicatively coupled to the patient device) that allows a user of the remote device to control the local camera hardware.

For example, a clinician may control a pan-and-tilt camera system attached to a patient device to focus on the feature of interest, control an aperture of the camera to address lighting and contrast issues, etc. In another example, the patient's proximity to the camera on the patient device may be detected from the captured video data, and the audio volume and/or microphone gain on the patient device may be modulated in real-time to ensure that the audio quality remains strong for both the patient and clinician. For example, if the patient moves away from the patient device to demonstrate walking for the clinician, the volume on the patient device may be increased to ensure clinician instructions remain clear, and the gain of the microphone on the patient device may be increased to ensure the patient's voice can still be heard by the clinician.

In another embodiment, a user may provide feedback on the captured field of view through a user interface that allows to user to tap on or otherwise select an area that has an inadequate resolution. Actions taken in response to the selection may vary depending on the system, desired use, or user intent. For example, in response to a user selecting a video location with a potential quality problem, machine learning analysis may be applied specifically to the selected location, improving system performance.

The system may additionally or alternatively respond to the selected video location by taking a pre-determined action, such as zooming in on the selected location. Those of skill in the art will appreciate that this type of response may be extended to address alternative problems with video, such as luminance or contrast problems. The response to selection of a video location may be defined in the remote therapy application and may be configurable by the manufacture or user, and/or a menu of response options may be presented to a user at the time of selection. The response may be implemented using software and/or hardware functionality, as appropriate.

Luminance

The relatively limited dynamic range of electronic camera systems as compared to the human eye may lead to situations where a person can see their environment adequately, but a camera system does not provide sufficient detail to remote users. That is, while an overall scene may be well lit, if the lighting does not sufficiently illuminate a feature of interest, then the luminance is inadequate.

For example, DBS stimulation may, in some cases, cause twitching or pulling of facial muscles when stimulation intensity is relatively high. If the patient's face is shaded, the clinician may be unable to perform an adequate assessment, despite otherwise adequate lighting.

Accordingly, machine learning algorithms that detect the patient's face may be used in the scenario. For example, machine learning analysis of the patient's face may be used to generate prompts displayed to the patient (e.g., “please ensure even lighting”, “please ensure the camera framing clearly includes your face”, etc.). Further, machine learning algorithms may provide improved targeting for threshold based luminance detection. In addition, machine learning algorithms may leverage hardware such as lighting sources or camera aperture controls (e.g., camera LEDS, USB ‘selfie rings’, electronically controllable camera lenses) to add lighting to the scene, or reduce light input to directly correct lighting deficiencies. Although the example give herein is a patient's face, those of skill in art will appreciate that these embodiments may be applied to any body part or luminance condition.

In one embodiment, the device (e.g., patient device 104 and/or clinician device 130) is capable of interfacing with one or more appliances and/or electronics (e.g., lighting fixtures) at the user's location. To improve lighting for the user, the device may automatically adjust the one or more appliances and/or electronics. For example, the device may interfaces with a lighting fixture to automatically increase or decrease the amount of light emitted by the lighting fixture as appropriate.

Contrast

Contrast is often an issue where natural lighting (e.g., from a window) creates bright spots in the camera's field of view. The limited dynamic range of a camera as compared to a human eye results in a naturally-lit area being displayed as well-lit, with the rest of the image being dark and washed-out. Alternatively, tight spot-lighting of one area of a video image may cause that region to be washed-out, and lacking in detail. Accordingly, machine learning algorithms may be applied using the systems and methods described herein to improve contrast as well.

Background Audio Noise

Background noise from devices or activities is often an issue in teleconferencing software. This may be a result of software settings that attempt to selectively amplify voices, resulting in exaggerated transmission of background chatter. Further, there may simply be ambient noise pollution. In addition to detection of echo between a speaker and a microphone, machine learning systems may be utilized to detect background features such as background chatter, multiple voices, music, etc.

Having identified a potential source of background audio noise, the system may provide more specific feedback to the user (e.g., displaying “it sounds like music is playing nearby, can you please turn that off or move to a quiet place for your telehealth session?”). In another embodiment, the speech of participants may be monitored (e.g., using speech to text and natural language processing) for statements that indicate audio issues, such as statements like “you are very quiet”, “speak up”, “please repeat that”, “I'm getting echo”, etc. By detecting these statements, the system may assist the user in addressing the issue by directly modifying the volume or gain of microphones, or by providing notifications and assistive controls (e.g. displaying “it sounds like you are having trouble hearing, press here to adjust your device volume”). In some cases, these approaches may be combined (e.g., when the permitted direct volume control alone is insufficient, the user may also be prompted to adjust the device's master volume).

Utilizing Feedback Control of Local Sensors to Effect Resolution of Identified Quality Problems

Once a quality issue with a telehealth session is identified, specific actions may be taken to resolve the issue. In the simplest case, the user may be informed, and allowed to take corrective action. This may be prompted by displays aimed to provide specific feedback on the extent of the problem. For example, a framing issue that cuts off an area of interest such as the patient's hands, might be highlighted by a glowing border at the edge of the displayed video image where the patient's hands are obscured. This glowing border may remain until the patient's hands are brought into frame.

In another example, a pop-up graphic depicting the extent of the issue is displayed with either a continuous scale, or discrete levels (e.g. “Poor”, “OK”, “Good”). Further, some systems may have access to controls to address the issues. Electronic cameras, for example, may be able to address luminance and contrast issues by adjusting the ISO settings or utilizing a High Dynamic Range (HDR) mode. Audio systems may be able to address feedback or low voice levels compared to background by adjusting the gain of the microphones and speakers on the telehealth device. Framing problems may be addressed by utilizing a pan-and-tilt camera, which allows for electronically controlled adjustment of the orientation of the camera in addition to the other camera settings.

Alternatively, in many cases, the local sensor (e.g., the sensor on the patient device) is of higher resolution than the transmitted resolution of the telehealth system, allowing for digital cropping to increase the effective resolution on an area of interest to address cases where the patient may be too far away from the camera, or where the field of view is too wide to properly highlight the area of interest. It is important to note that zooming and cropping that is performed on the source device (e.g., the patient device) is typically superior to cropping and zooming on the viewing device (e.g., the clinician device), as the source device has a higher resolution stream to work with, whereas the stream transmitted to the viewing device is of lower resolution, and therefore contains less detail to elucidate by cropping and digital zooming.

Two alternative methods of assisting the user include providing machine learning systems capable of identifying the features of interest of the patient, and providing the clinician with a score or report to aid in their assessment of the lower quality transmitted video. Alternatively, the system could trigger local recording of high resolution video, which is then made available to the other user (e.g., via upload to a cloud system, or by direct transfer between the devices). This allows the clinician to review higher resolution videos to refine their assessments. The transmission of video files in parallel with the telehealth video sessions may include relatively small video files within the same session, allowing the clinician to immediately review something in finer detail, or may be larger video files stored for review in planning future therapy adjustments, adding detail to clinical notes, attaching to EMR records etc.

In one embodiment, a user (e.g., the patient or clinician) provides an assessment of the video quality. In response, the system can automatically adjust the video resolution to attempt to address any issues. For example, if a user indicates the video quality is lagging or jumping, the system may lower the video resolution. In contrast, if a user indicates the video quality is unclear or hard to see, the system may increase the video resolution.

Specialized Machine Learning Algorithms that Provide Measures of Patient or Symptom Status During the Session

In some embodiments, machine learning algorithms may be utilized to detect a patient status, or other events, and to notify the clinician to ensure the clinician does not miss an event of interest or to provide additional detail for the clinician's evaluation. If these algorithms are in use during a telehealth session, they may be employed prior to the session, and the output of these algorithms may be utilized to inform quality decisions and prompt performed by the system.

An example of this is machine learning systems that use a video of the patient's face to compute the user's heart rate, and blood pressure. These algorithms utilize the relative transmittance of red and green light through skin perfused with oxygenated blood, and unperfused skin to evaluate the occurrence of each heartbeat. Since these algorithms are reliant on relative changes in specific colored light intensities within the video, the output of such a machine learning network could also be used to inform the user in how to optimize lighting for the performance of such a machine learning algorithm.

Network Connectivity

Telehealth systems generally rely on network connections to transmit audio, video, and any therapy data between the participants. Since audio and video data in particular are sensitive to bandwidth and latency, the quality of the connection is relevant to the quality of the resulting telehealth session. Upon initial connection to a supporting cloud infrastructure, or to the device of the other participants (as shown in FIG. 1), the system may transmit trial data in order to measure the bandwidth and latency of transmissions between components. This allows the system to present the user with, for example, an empirical measure of bandwidth or latency, or a qualitative indicator of the network status (e.g., “Slow”, “OK”, “Fast”; or “Poor”, “OK”, “Good”). If the status is poor (i.e., low bandwidth or high latency) the system may provide additional recommendations to the user, such as displaying “ensure you are close enough to your WiFi to get a strong signal” or “check to see whether other users or devices may be monopolizing your internet connection”. Additional embodiments contemplate separately connecting each user device to a centralized cloud service, enabling users to identify which component may be having network connection issues.

Test Mode

In some embodiments, a test mode is utilized to prompt the user (e.g., the patient) to perform specific actions designed to identify quality issues. That is, the user may be prompted to perform specific actions in order to identify problems with greater precision.

For machine learning algorithms, it is generally the case that the less constrained a data set, the more difficult it is to train the machine learning network. By prompting the user to perform specific actions, the system may employ a specialist network created to identify the specific action the user is performing. This provides greater sensitivity, and therefor will generally improve the system's ability to detect and respond to potential quality problems.

This may provide additional benefits in scenarios where the task the user is asked to perform is part of a standard evaluation that the telehealth system is designed to provide assistive metrics on. This is useful, as it may streamline the visit by providing the clinician with appropriate data prior to the initiation of the actual telehealth session.

In other scenarios, these baseline measurements may be of greater utility, as a telehealth session may be used to adjust the patient's therapy (e.g., by adjusting stimulation parameters of an implanted device), and the before and after measurement of basic clinical tests may be utilized to evaluate the efficacy of the adjustments. For example, a standard evaluative test performed for Parkinson's disease patients is a simple rapid pinching task, where the patient is asked to hold their hand up, and to bring their index finger and thumb together and apart as rapidly as possible. The evaluating clinician then utilizes the frequency, maximum amplitude of the opening between fingers, variability in time between closings, etc. to make an assessment of the status of the patient's motor symptoms. A telehealth system designed for DBS devices in the Parkinsonian population might provide assistive analytics of a finger tapping task (where patients tap their index finger to their thumb repeatedly) such as frequency, variability, aperture between finger and thumb, etc., and performing this pinching test prior to the session may serve the dual purpose of providing a baseline clinical measure, as well as providing feedback on the quality of the video.

Any standard or common clinical test may be treated in this manner, and provide a baseline measure of the patient's performance in that task. These measures might use validated clinical scales if sufficient evidence is available to demonstrate that the algorithm matches the performance of clinical experts, or might be a customized score that provides an additional feedback point for the clinician.

Examples of a test mode include asking the patient to smile at the camera, frown at the camera, blink at the camera, stick their tongue out, look left then right, etc. Actions involving the face enable both simple machine learning networks that are adept at identifying faces to locate the user in the telehealth video stream, and more specialized machine learning networks to identify the various facial actions. The ability to identify deliberate facial actions such as smiling, frowning, blinking etc. can be a proxy for the quality of the video conveying facial symptoms to the clinician. This also allows the machine learning algorithm to identify whether the user's face is in the camera's field of view, and to provide guidance on centering if it is near the edge of frame. Similarly, the system may identify what portion of the screen is taken up by the face, and recommend either zooming in or out, or moving the camera further from or closer to the patient to optimize the size of the face in the video. Finally, identification of the arms allows the system to evaluate luminance and contrast of the portion of video occupied by the face, providing specific feedback on those aspects of video quality for the face.

Other examples include asking the patient to wave to the camera, hold their hands out in front of them, clap for the camera, point from their nose to the camera, etc. Actions involving the hands and arms enable machine learning networks that are adept at identifying arms and hands to locate the user's hands in the field of view. The ability to evaluate positions of the arms may be used to evaluate motor symptoms such as tremor, dyskinesia, dystonia, hypertonicity, etc. The certainty with which a network can identify the position of arms and hands may provide a proxy for the quality with which the telehealth video session conveys the position of the arms or hands.

Further, actions such as clapping allow synchronization of an audio event with the video to establish the fidelity with which the video conveys rapid events. This is especially critical for evaluation of rhythmic symptoms such as tremor or phasic dyskinesia as video compression algorithms using key-frame compression can drop the excursions from motions, causing the arm to appear relatively stable in video when the tremor is readily evident when observed in-person. These tests also enable the machine leaning algorithms to identify when the arms and hands are in the field of view, allowing the system to provide feedback to help the user adjust the orientation and location of the camera to better capture the upper limbs. Similarly, the network may identify what portion of the screen is taken up by the upper limb, and recommend either zooming in or out, or moving the camera further or closer to optimize the size of the arm in the video. Finally, identification of the arms allows the system to evaluate luminance and contrast of the portion of video occupied by the arm, providing specific feedback on those aspects of video quality for the arm. Pointing from the nose to a fixed point repeatedly is a commonly used clinical test for movement disorders patients such as Parkinson's disease patients, and is used in the Unified Parkinson's Disease Rating Scale (UPDRS) evaluation (subscale 3), where performance is ranked on a scale from 0-4 scale.

Other examples include asking the patient to perform a finger tapping test for camera, hold up their fingers one at a time as quickly as possible, tap the table as quickly as possible, etc. Actions involving the hand and fingers enable analysis by machine learning networks that are adept at identifying hand and finger location and position. This provides feedback on the quality of the video conveying the user's hand. Simple tests such as finger tapping or pinching are often used to evaluate symptoms in movement disorder patients, and may be more generally used to evaluate manual dexterity. These tests also enable the machine leaning algorithms to identify when the hands are in the field of view, allowing the system to provide feedback to help the user adjust the orientation and location of the camera to better capture the hands. Similarly, the machine learning networks may identify what portion of the screen is taken up by the hand, and recommend either zooming in or out, or moving the camera further or closer to optimize the size of the hand in the video. Finally, identification of the hands allows the system to evaluate luminance and contrast of the portion of video occupied by the hand, providing specific feedback on those aspects of video quality for the hand. Finger tapping in particular is a commonly used clinical test for movement disorders patients such as Parkinson's disease patients, and is used in the UPDRS evaluation (subscale 3), where performance is ranked on a 0-4 scale.

Other examples include asking the patient to stand up or sit down, tap their foot on the floor, tap their heel on the floor, etc. Actions involving the feet enable applying machine learning algorithms specialized in detecting the location, orientation, and joint configurations of the feet. This provides feedback on the quality of the video conveying the user's feet. Simple tests such as foot tapping may be utilized when evaluating motor disorders or coordination that impacts the user's lower limbs. These tests also enable the machine leaning algorithms to identify when the feet are in the field of view, allowing the system to provide feedback to help the user adjust the orientation and location of the camera to better capture the feet. Similarly, the machine learning algorithms may identify what portion of the screen is taken up by the feet, and recommend either zooming in or out, or moving the camera further or closer to optimize the size of the legs and feet in the video. Finally, identification of the feet allows the system to evaluate luminance and contrast of the portion of video occupied by the feet, providing specific feedback on those aspects of video quality for the feet.

As yet another example, the system may ask the patient to walk away from the camera (e.g., 10 steps) and return. Walking enables analysis by machine learning algorithms specialized in detecting the location orientation and joint configuration of the legs and feet. This provides feedback on the quality of the video conveying the user's legs. Simple walking tests are frequently used to assess balance or gait disturbances which may be caused by a variety of conditions. These tests also enable the machine leaning algorithms to identify when the legs and feet are in the field of view, allowing the system to provide feedback to help the user adjust the orientation and location of the camera to better capture the legs and feet. Similarly, the network may identify what portion of the screen is taken up by the legs and feet, and recommend either zooming in or out, or moving the camera further or closer to optimize the size of the legs and feet in the video. Finally, identification of the legs and feet allows the system to evaluate luminance and contrast of the portion of video occupied by the legs and feet, providing specific feedback on those aspects of video quality for the legs and feet. Machine learning algorithms may also explicitly evaluate clinical scores such as the UPDRS scores for walking difficulty, freezing when walking, and gait assessments.

As further examples, the system may ask the patient to speak a particular sentence or phrase, clap their hands, snap their fingers, etc. Utilizing known sentences or phrases allows the use of speech-to-text algorithms and provides both the opportunity for utilizing specialized machine learning algorithms, as well as knowledge of what the precise output should be, providing an ideal reference for the quality of the algorithm performance. This provides dedicated feedback on the quality with which the user's voice is conveyed to the other party. This data may be combined with data on the intensity of the sound picked up by the system microphone to provide specific feedback: if intensity is high, but algorithm performance is low, this indicates that there may be background noise, and the user should be prompted to reduce noise, or move to a different location. If additional data is desired, the user might be prompted to refrain from speaking (e.g., for 10 seconds) to evaluate the intensity of noise when the user is not speaking. If the intensity of the sound is low, the user may be prompted to move closer to the microphone, or adjust the gain of their audio device. Alternatively the user may be informed that they are not clear, and that they may need to speak louder. This may be especially important for certain conditions such as Parkinson's disease that may cause hypophonia, or populations such as the elderly who may be prone to weaker voices.

As another example, the system may play pre-recorded speech and ask the patient to confirm they can clearly hear the audio. Utilizing pre-recorded speech allows the user to provide feedback on whether the volume of the device is adequate. The user may be provided with an interface to either respond verbally, or a computerized interface where they may indicate if the audio is too loud, too quiet, unclear, or just right. This feedback allows the system to either directly modify the speaker settings of the user's device, or to provide assistance (e.g., displaying “tap here to go to your device's volume control”).

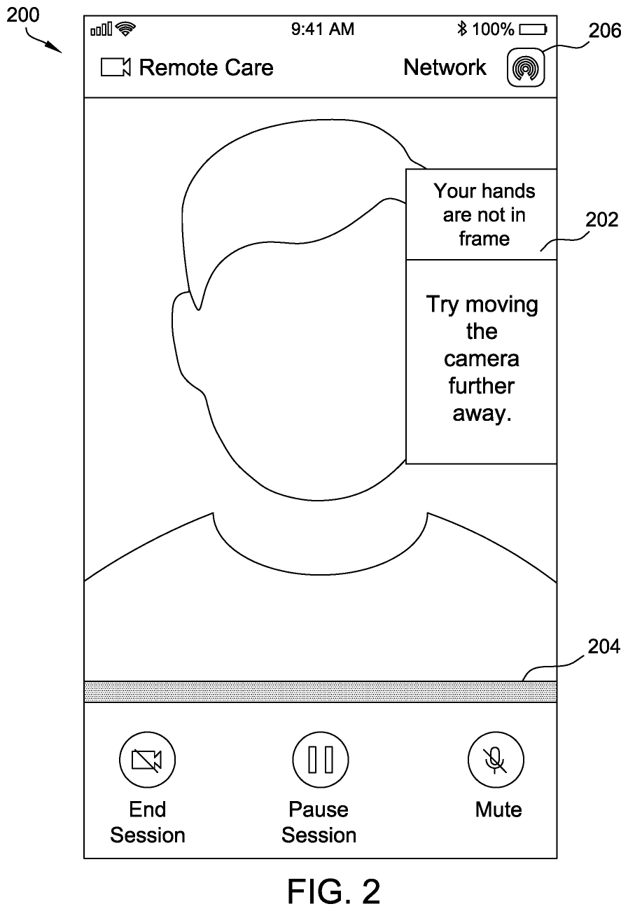

FIGS. 2-5 are example user interfaces that may be displayed, for example on patient device 104 or clinician device 130 (both shown in FIG. 1).

FIG. 2 shows one embodiment of a user interface 200 (e.g., to be displayed to patient 102). User interface 200 includes an alert 202 notifying the user that their hands are not in frame, and also includes highlighting 204 at the bottom of the frame to indicate which direction the field of view should be adjusted in. User interface 200 also includes an indicator 206 demonstrating the status of the network connection. In this example, the depicted embodiment of the network status indicator shows an assessment of the connection to a cloud service prior to initiation of a telehealth session.

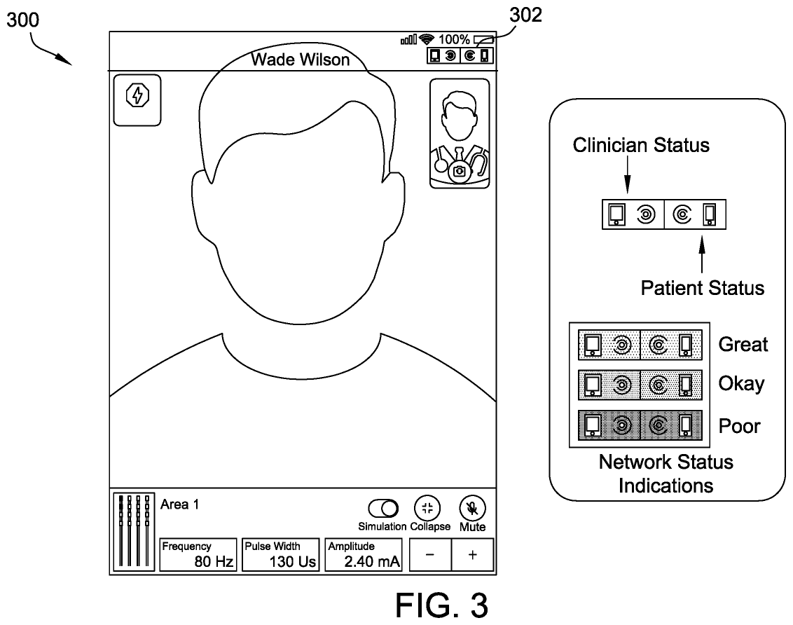

FIG. 3 shows another embodiment of a user interface 300 (e.g., to be displayed to clinician 138). User interface 300 may be displayed, for example, as part of a telehealth session involving an implantable spinal cord generator. User interface 300 includes a network indicator 302 which depicts the connectivity status of both the clinician device and the patient device. The user interface 300 provides continuous monitoring of the network status during the telehealth session.

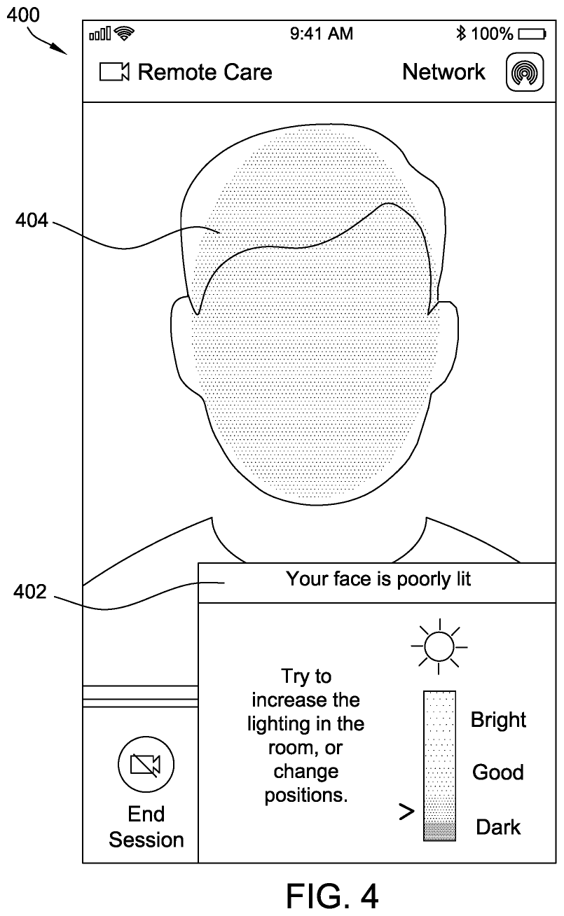

FIG. 4 shows another embodiment of a user interface 400 (e.g., to be displayed to patient 102). User interface 400 includes a notification 402 telling the user there is a luminance issue with their face, and also graphically shows they user that their face is shaded by displaying a partially transparent object 404.

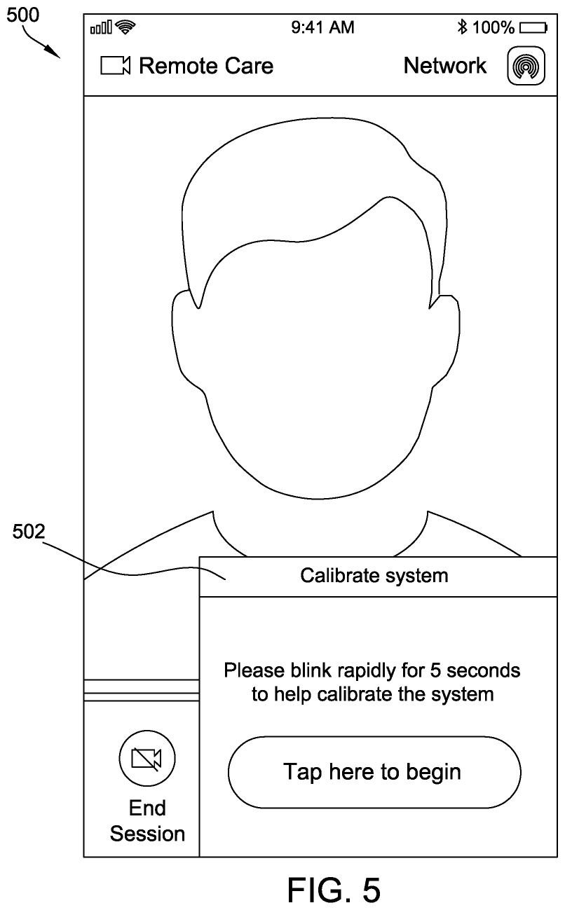

FIG. 5 shows another embodiment of a user interface 500 (e.g., to be displayed to patient 102). User interface 500 includes a prompt 502 telling the user to perform an action (e.g., blink rapidly) to assist in evaluating the video quality.

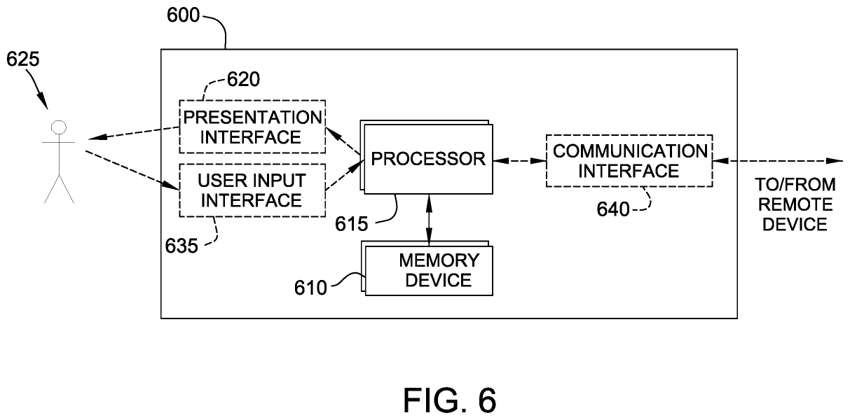

FIG. 6 illustrates one embodiment of a computing device 600 that may be used to implement the systems and methods described herein. For example, computing device 600 may be used to implement patient device 104 and/or clinician device 130 (bot shown in FIG. 1).

Computing device 600 includes at least one memory device 610 and a processor 615 that is coupled to memory device 610 for executing instructions. In some embodiments, executable instructions are stored in memory device 610. In this embodiment, computing device 600 performs one or more operations described herein by programming processor 615. For example, processor 615 may be programmed by encoding an operation as one or more executable instructions and by providing the executable instructions in memory device 610.

Processor 615 may include one or more processing units (e.g., in a multi-core configuration). Further, processor 615 may be implemented using one or more heterogeneous processor systems in which a main processor is present with secondary processors on a single chip. In another illustrative example, processor 615 may be a symmetric multi-processor system containing multiple processors of the same type. Further, processor 615 may be implemented using any suitable programmable circuit including one or more systems and microcontrollers, microprocessors, reduced instruction set circuits (RISC), application specific integrated circuits (ASIC), programmable logic circuits, field programmable gate arrays (FPGA), and any other circuit capable of executing the functions described herein. In one embodiment, processor 615 is a GPU (as opposed to a central processing unit (CPU)). Alternatively, processor 615 may be any processing device capable of implementing the systems and methods described herein.

In this embodiment, memory device 610 is one or more devices that enable information such as executable instructions and/or other data to be stored and retrieved. Memory device 610 may include one or more computer readable media, such as, without limitation, dynamic random access memory (DRAM), static random access memory (SRAM), a solid state disk, and/or a hard disk. Memory device 610 may be configured to store, without limitation, application source code, application object code, source code portions of interest, object code portions of interest, configuration data, execution events and/or any other type of data. In one embodiment, memory device 610 is a GPU memory unit. Alternatively, memory device 610 may be any storage device capable of implementing the systems and methods described herein.

In this embodiment, computing device 600 includes a presentation interface 620 that is coupled to processor 615. Presentation interface 620 presents information to a user 625 (e.g., patient 102 or clinician 138). For example, presentation interface 620 may include a display adapter (not shown) that may be coupled to a display device, such as a cathode ray tube (CRT), a liquid crystal display (LCD), an organic LED (OLED) display, and/or an “electronic ink” display. In some embodiments, presentation interface 620 includes one or more display devices.

In this embodiment, computing device 600 includes a user input interface 635. User input interface 635 is coupled to processor 615 and receives input from user 625. User input interface 635 may include, for example, a keyboard, a pointing device, a mouse, a stylus, a touch sensitive panel (e.g., a touch pad or a touch screen), a gyroscope, an accelerometer, a position detector, and/or an audio user input interface. A single component, such as a touch screen, may function as both a display device of presentation interface 620 and user input interface 635.

Computing device 600, in this embodiment, includes a communication interface 640 coupled to processor 615. Communication interface 640 communicates with one or more remote devices. To communicate with remote devices, communication interface 640 may include, for example, a wired network adapter, a wireless network adapter, and/or a mobile telecommunications adapter.

The embodiments described herein provide systems and methods for improving quality of a remote therapy session. A method includes capturing data associated with a remote therapy session between a patient device and a clinician device, applying one or more machine learning algorithms to the captured data to detect a quality issue associated with the remote therapy session, and performing a remedial action to address the detected quality issue.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A method for improving quality of a remote therapy session, the method comprising:
capturing, using a computing device, video data associated with a remote therapy session between a patient device and a clinician device;
applying, using the computing device, one or more machine learning algorithms to the captured video data to detect a quality issue associated with the remote therapy session, the quality issue related to one of i) a field of view associated with the video data, ii) a luminance associated with the video data, and iii) a contrast associated with the video data; and
performing, using the computing device, a remedial action to address the detected quality issue in the video data.

2. The method of claim 1, wherein applying one or more machine learning algorithms to the captured video data comprises applying one or more machine learning algorithms to determine whether the video data shows a feature of interest within the field of view.

3. The method of claim 3, wherein performing a remedial action comprises generating a notification to alert a user of the patient device that the field of view should be adjusted.

4. The method of claim 1, wherein the computing device is one of the patient device and the clinician device.

5. The method of claim 1, wherein performing a remedial action comprises displaying a prompt on one of the patient device and the clinician device.

6. The method of claim 1, wherein performing a remedial action comprises controlling a camera on one of the patient device and the clinician device.

7. The method of claim 1, wherein capturing data comprises capturing data during a test mode prior to initiation of the remote therapy session.

8. A computing device for improving quality of a remote therapy session, the computing device comprising:
a memory device; and
a processor communicatively coupled to the memory device, the processor configured to:
capture data associated with a remote therapy session between a patient device and a clinician device;
apply one or more machine learning algorithms to the captured data to detect a quality issue associated with the remote therapy session; and
perform a remedial action to address the detected quality issue.

9. The computing device of claim 8, wherein to capture data, the processor is configured to capture video data.

10. The computing device of claim 9, wherein to apply one or more machine learning algorithms, the processor is configured to apply one or more machine learning algorithms to detect a quality issue related to one of i) a field of view associated with the video data, ii) a luminance associated with the video data, and iii) a contrast associated with the video data.

11. The computing device of claim 8, wherein to capture data, the processor is configured to capture audio data.

12. The computing device of claim 11, wherein to apply one or more machine learning algorithms, the processor is configured to apply one or more machine learning algorithms to detect a quality issue related to background noise associated with the audio data.

13. The computing device of claim 8, wherein to perform a remedial action, the processor is configured to at least one of i) display a prompt on one of the patient device and the clinician device, ii) control a camera on one of the patient device and the clinician device, and iii) control an audio system on one of the patient device and the clinician device.

14. The computing device of claim 8, wherein to capture data, the processor is configured to capture data during a test mode prior to initiation of the remote therapy session.

15. The computing device of claim 8, wherein the computing device comprises the patient device.

16. The computing device of claim 8, wherein the computing device comprises the clinician device.

17. Non-transitory computer-readable media having computer-executable instructions thereon, wherein when executed by a processor of a computing device, cause the processor of the computing device to:
capture data associated with a remote therapy session between a patient device and a clinician device;
apply one or more machine learning algorithms to the captured data to detect a quality issue associated with the remote therapy session; and
perform a remedial action to address the detected quality issue.

18. The non-transitory computer-readable media of claim 17, wherein to capture data, the instructions cause the processor to capture video data.

19. The non-transitory computer-readable media of claim 17, wherein to capture data, the instructions cause the processor to capture audio data.

20. The non-transitory computer-readable media of claim 17, wherein to perform a remedial action, the instructions cause the processor to at least one of i) display a prompt on one of the patient device and the clinician device, ii) control a camera on one of the patient device and the clinician device, and iii) control an audio system on one of the patient device and the clinician device.
